(19) United States
(12) Patent Application Publication
(10) Pub. No.: US 2024/0054774 A1
(43) Pub. Date: Feb. 15, 2024

(54) METHOD OF PROVIDING USER PROPENSITY ANALYSIS SERVICE USING ARTIFICIAL INTELLIGENCE-BASED FINGERPRINTS

(71) Applicant: WAVE3D CO., LTD., Jeollanam-do (KR)

(72) Inventor: Kyung-Sik Seo, Gwangsan-gu (KR)

(21) Appl. No.: 18/058,248

(22) Filed: Nov. 22, 2022

(30) Foreign Application Priority Data
Aug. 10, 2022 (KR) 10-2022-0099759

(51) Int. Cl. G06V 10/82; G06V 40/12; G06V 10/774; G06Q 10/10

(52) U.S. Cl. CPC G06V 10/82; G06V 40/1359; G06V 10/774; G06Q 10/1053

(57) ABSTRACT

A method of providing user propensity analysis service using artificial intelligence-based fingerprints includes: generating a learning model for determining fingerprint types, configured to collect data samples including fingerprints through an execution of a program in a computing device, and determine at least 12 types of fingerprints through image analysis and artificial intelligence learning on the collected data samples; collecting data adapted to determine propensities of the fingerprints, then establishing determination data for determining the propensities for each of at least 12 or more types of fingerprints based on the determined propensities; when a fingerprint image of the user is input, applying the input fingerprint image to the learning model for determining the fingerprint type to determine the fingerprint type of the user; and generating user propensity information using the determined data for the fingerprint type and providing the user propensity information to the user.

### CROSS REFERENCE TO RELATED APPLICATION

This application claims priority benefit of Korean Patent Application No. 10-2022-0099759 filed on Aug. 10, 2022, the entirety of which is incorporated by reference herein.

### BACKGROUND OF THE INVENTION

### 1. Field of the Invention

The present invention relates to a propensity analysis using fingerprints of a user based on artificial intelligence.

### 2. Description of the Related Art

Efforts to figure out innate propensities and abilities of a human by analyzing fingerprints thereof have continued throughout human history. Recently, efforts have been made to perform aptitude and multitasking ability tests of a human by analyzing fingerprint information of ten fingers.

However, the conventional aptitude test and multitasking ability (intelligence) test have only provided insights into behavioral superiority, and there has been no effort to identify an accurate career path by using the test results. Accordingly, in determining the career path, since the test results cannot be used even after taking a test, many people have realized difficulties to find an appropriate career path, and have been experiencing difficulties to accurately evaluate the value of a genetic fingerprint aptitude test.

In addition, although characteristics of individual propensities were identified, since there was no additional study on countermeasures for each propensity, many difficulties have arisen for efficiently utilizing the genetic fingerprint aptitude test.

Further, there is a case where a congenital fingerprint is slightly changed due to some life event or environmental factors, and in this case, it is difficult to accurately recognize a fingerprint pattern according to such a slight change.

### PRIOR ART DOCUMENT

### Patent Document

(Patent Document 0001) Korean Patent Laid-Open Publication No. 10-2022-0022592 (published on Feb. 28, 2022)

### SUMMARY OF THE INVENTION

It is an object of the present invention to provide a method of providing a user propensity analysis service using artificial intelligence-based fingerprints, which includes generating a learning model capable of determining a fingerprint type using artificial intelligence, then determining a type of the input fingerprint based on the generated learning model, and providing user propensity information according to the determined fingerprint type, thereby accuracy in determining the type of fingerprint can be increased.

To achieve the above object, according to an aspect of the present invention, there is provided a method of providing user propensity analysis service using artificial intelligence-based fingerprints, wherein the user propensity is analyzed and provided to a user based on the fingerprints by a program which is executed by at least one or more processors and stored in a computing device, the method including: generating a learning model for determining fingerprint types, which is configured to collect data samples including fingerprints through an execution of the program in the computing device, and determine at least 12 types of fingerprints through image analysis and artificial intelligence learning on the collected data samples; collecting data adapted to determine propensities in relation to the fingerprints, and then establishing determination data for determining the propensities for each of at least 12 or more types of fingerprints based on the determined propensities; when a fingerprint image of the user is input, applying the input fingerprint image to the learning model for determining the fingerprint type to determine the fingerprint type of the user; and generating user propensity information using the determined data for the fingerprint type and providing the user propensity information to the user.

According to an embodiment of the present invention, the step of generating a learning model for determining the fingerprint type may include: classifying the data samples into at least 12 or more types of fingerprints; generating a training dataset for each fingerprint type by propagating the data samples through various changes to the classified data samples of each fingerprint type; performing CNN training on the training dataset of each fingerprint type to extract feature points for each fingerprint type, and generating a pattern of each fingerprint type through statistical analysis of the extracted feature points; and when the fingerprint image of the user is input, generating a model for determining the fingerprint type, which is adapted to determine the fingerprint type of the user as any one of the 12 or more types of fingerprints using the pattern.

According to an embodiment of the present invention, the computing device may include a first relational database adapted to compare relationships between various propensities and a second relational database adapted to compare propensities with characteristics of job and occupational groups, and the step of providing the user propensity information to the user may include generating a report further including information on jobs and occupational groups suitable for the propensity of the user through linkage analysis between the user propensity information and data stored in the first and second relational databases, by using a relationship between the user and a user having a different propensity from the user.

According to an embodiment of the present invention, the 12 types of fingerprints may include at least one of a Radial Loop, Whorl, and a Double Loop patterns, and the step of generating a model for determining the fingerprint type may include: classifying the data samples into at least 15 types of fingerprints in a manner of: calculating a degree of deformation through comparison between reference shape data for the Radial Loop and the fingerprint images in the data samples, and then if the degree of deformation is out of a preset threshold range, determining the fingerprint as a deformed Radial Loop; calculating a degree of deformation through comparison between reference shape data for the Whorl and the fingerprint images in the data samples, and then if the degree of deformation is out of the preset threshold range, determining the fingerprint as a deformed Whorl; and calculating a degree of deformation through comparison between reference shape data for the Double Loop and the fingerprint images in the data samples, and then if the degree of deformation is out of the preset threshold range, determining the fingerprint as a deformed Double Loop; generating a training dataset for each fingerprint type by propagating the data samples through various changes to the classified data samples of each fingerprint type; performing CNN training on the training dataset of each fingerprint type to extract feature points for each fingerprint type, and generating a pattern of each fingerprint type through statistical analysis of the extracted feature points; and when the fingerprint image of the user is input, generating a model for determining the fingerprint type, which is adapted to determine the fingerprint type of the user as any one of the 15 or more types of fingerprints using the pattern.

According to the above-described embodiments of the present invention, by generating a learning model capable of determining a fingerprint type using artificial intelligence, then determining a type of the input fingerprint based on the generated learning model, and providing user propensity information according to the determined fingerprint type, accuracy in determining the type of fingerprint can be increased, and thereby, it is possible to increase the accuracy of user propensity determination using the fingerprint.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram illustrating a configuration of a computing device to which general artificial intelligence technologies are applied;

FIG. 2 is a block diagram illustrating a detailed configuration of the computing device capable of providing a method of providing a user propensity analysis service according to an embodiment of the present invention;

FIG. 3 is a flowchart illustrating a process of analyzing fingerprint-based user propensity information according to an embodiment of the present invention;

FIG. 4 is a diagram illustrating a configuration of a fingerprint applied to an embodiment of the present invention; and

FIG. 5 is an exemplary view for describing a report format provided by an artificial intelligence program according to an embodiment of the present invention.

### DETAILED DESCRIPTION OF THE INVENTION

Hereinafter, specific embodiments of the present invention will be described with reference to the accompanying drawings. The following detailed description is provided to contribute to a comprehensive understanding of a method, apparatus, and/or system described in the present disclosure. However, these embodiments merely illustrative examples, and the present invention is not limited thereto.

In descriptions of the embodiments of the present invention, publicly known techniques that are judged to be able to make the purport of the present invention unnecessarily obscure will not be described in detail. Referring to the drawings, wherein like reference characters designate like or corresponding parts throughout the several views. In addition, the terms as used herein are defined by taking functions of the present disclosure into account and can be changed according to the custom or intention of users or operators. Therefore, definition of the terms should be made according to the overall disclosure set forth herein. In addition, the terminology used herein is for the purpose of describing particular embodiments only and is not intended to limit the present invention thereto. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Hereinafter, a computing device for providing an artificial intelligence-based user propensity analysis service according to an embodiment of the present invention and a method of providing a user propensity analysis service using the same will be described with reference to the accompanying drawings.

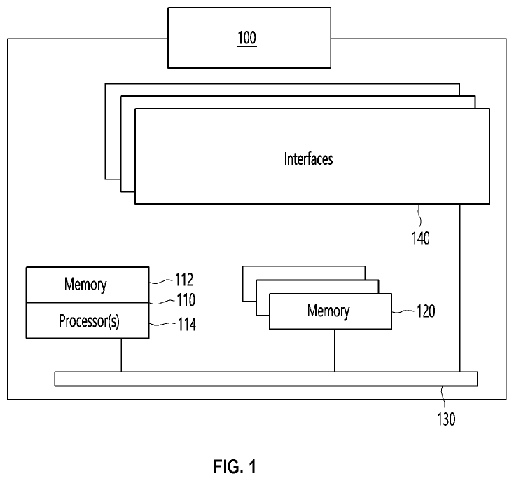

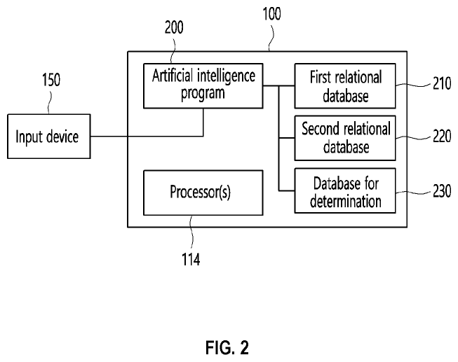

FIG. 1 is a block diagram illustrating a configuration of a computing device to which general artificial intelligence technologies are applied, and FIG. 2 is a block diagram illustrating a detailed configuration of the computing device capable of providing a method of providing a user propensity analysis service according to an embodiment of the present invention.

As shown in FIG. 1, a computing device 100 to which artificial intelligence technologies are applied in the embodiment of the present invention includes a central processing unit (CPU) 110, interfaces 140, and a bus 130 (such as a peripheral component interconnect (PCI), etc.).

When operating the computing device under the control of appropriate software or firmware, the CPU 110 may implement certain functions associated with the functions of a specifically configured computing device or machine. For example, in the embodiment of the present invention, a server may be configured or designed to function as an artificial intelligence program system which utilizes the CPU 110, memories 120, and interface(s) 140. Further, in the embodiment of the present invention, it may allow the CPU 110 to execute the functions and/or tasks of one or more different types of artificial intelligence programs under the control of software modules/components (for example, including an operating system and any appropriate application software, drivers, etc.).

The CPU 110 may include, for example, one or more processor(s) 114 such as a Motorola processor, an Intel microprocessor family or a MIPS microprocessor family. In the embodiment of the invention, the processor(s) 114 may include hardware specifically designed to control the operations of the computing device 100 (for example, application-specific integrated circuits (ASICs), electrically erasable programmable read-only memories (EEPROMs), field-programmable gate arrays (FPGAs) and the like). In addition, the memory 120 such as a non-volatile random access memory (RAM) and/or read-only memory (ROM) also forms a part of the CPU 110. However, there are many different ways in which the memory can be coupled with the system. Memory blocks 112 and 120 may be used for various purposes, such as caching and/or storage of data, and programming instructions, etc., for example.

As used in the present disclosure, the term “processor” is not limited to just those integrated circuits referred to as a processor in the art, but rather widely refers to a microcontroller, a microcomputer, a programmable logic controller, an application-specific integrated circuit (ASIC), and any other programmable circuit.

In the embodiment of the invention, the interfaces 140 are provided as interface cards (sometimes referred to as “line cards”). The interface cards control the transmission and reception of data packets on a computing network, and support other peripheral devices which are sometimes used with the computing device 100. Among the interfaces 140 capable of being provided, there are Ethernet interfaces, frame relay interfaces, cable interfaces, DSL interfaces, token ring interfaces and the like. In addition, for example, various types of interfaces may be provided, such as a universal serial bus (USB), serial, Ethernet, FireWire, PCI, Parallel, radio frequency (RF), Bluetooth, near field communication (for example, using near field magnetics), 802.11 (WiFi), frame relay, TCP/IP, ISDN, fast Ethernet interfaces, gigabit Ethernet interfaces, asynchronous transfer mode (ATM) interfaces, high speed serial interfaces (HSSIs), point of sale (POS) interfaces, fiber data distributed interfaces (FDDIs) and the like.

These interfaces 140 may include ports suitable for communication with appropriate media. In some cases, these interfaces may also include an independent processor, and in some instances, may include volatile and/or non-volatile memories (for example, RAM).

The system shown in FIG. 1 represents one specific architecture for the computing device 100 to implement the inventive technique described in the present disclosure. However, this is by no means the only device architecture in which at least some of the features and techniques described in the present disclosure can be implemented. For example, architectures having one or any number of processor(s) 114 may be used, and such processor(s) 114 may be present within one device or distributed between any number of devices. In one embodiment, one processor 114 handles the communication together with routing calculation. In various embodiments, different types of features and/or functions of artificial intelligence programs may be implemented in an artificial intelligence program system, which includes server system(s).

Regardless of the network device configuration, the system of the present invention may employ one or more memories or memory modules (e.g., such as memory blocks), which are configured to store data, program instructions for general purpose network operations, and/or other information related to the functions of the program techniques for artificial intelligence described in the present disclosure. The program instructions may, for example, control the operations of an operating system and/or one or more application programs. The memory or memories may also be configured to store data structures, keyword classification information, advertisement information, user click and impression information, and/or other specific non-program information described in the present disclosure.

Such information and program instructions may be employed to implement the systems/methods described in the present disclosure. Therefore, at least some of embodiments relating to the network device may include a nontransitory machine-readable storage medium. The nontransitory machine-readable storage medium may be configured or designed to store, for example, program instructions, state information, and the like for executing various tasks described in the present disclosure. Examples of such nontransitory machine-readable media include magnetic media such as hard disks, floppy disks, and magnetic tape; optical media such as CD-ROM disks; magneto-optical media such as floptical disks, and hardware devices such as read-only memory (ROM) devices, flash memory, memristor memory, random access memory (RAM), and the like, which are specially configured to store and execute program instructions, but they are not limited thereto. Examples of the program instructions include both machine codes such as a code generated by a compiler, and files including a higher-level code that can be executed by a computer using an interpreter.

Meanwhile, the computing device 100 having the configuration as described above may be interworked with an input device 150 capable of receiving a user input, that is, a user fingerprint, as shown in FIG. 2.

An artificial intelligence program 200 according to an embodiment of the present invention executed on the computing device 100 provides a service which may: generate a model for determining a type of fingerprint through deep learning (for example, CNN) for at least 12 or more types of fingerprints using a plurality of data samples; and determine what the type of user fingerprint input through the input device 150 is based on the generated learning model for determining the fingerprint type; then analyze and provide user propensity to the user based on the determined fingerprint type.

In addition, the artificial intelligence program 200 executed in the computing device 100 according to an embodiment of the present invention may provide various additional information related to the user propensities through interworking with various databases, for example, first and second relational databases 210 and 220.

Hereinafter, a process of analyzing and providing a user propensity using the user input, i.e., a fingerprint image, by the artificial intelligence program 200 executed in the computing device 100 as described above will be described.

Prior to the description, the artificial intelligence program 200 executed by the computing device 100 is stored in the memories 120 and 112 in a form executable by at least one or more processors 114, and may be executed by at least one or more processors 114 when the user fingerprint is input from the input device 150.

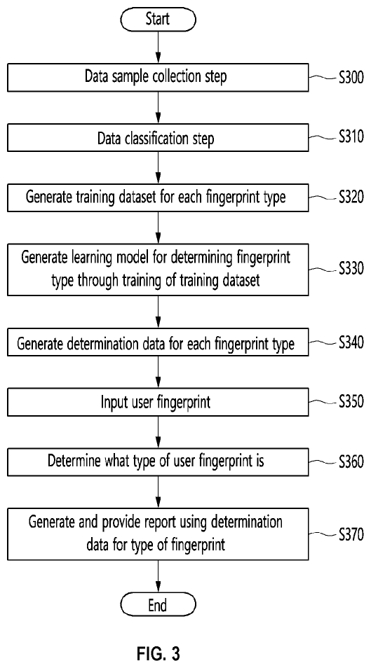

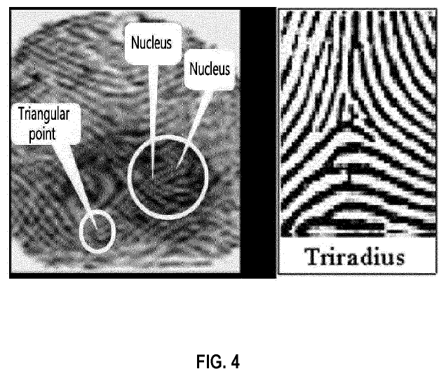

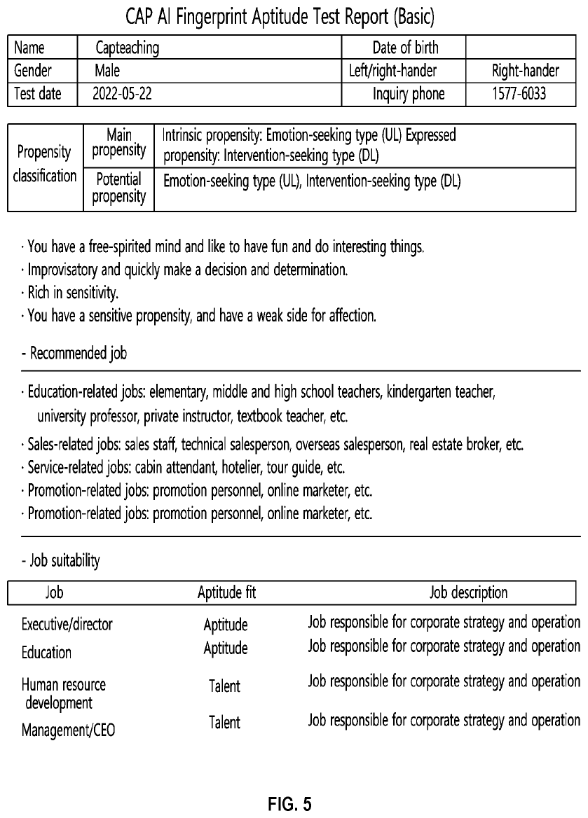

FIG. 3 is a flowchart illustrating a process of analyzing fingerprint-based user propensity information according to an embodiment of the present invention, FIG. 4 is a diagram illustrating a configuration of a fingerprint applied to an embodiment of the present invention, and FIG. 5 is an exemplary view for describing a report format provided by the artificial intelligence program according to an embodiment of the present invention.

Prior to the description of the embodiment of the present invention, the user propensity information may include personalities, temperament, qualities and the like.

As shown in FIG. 3, the process of analyzing fingerprint-based user propensity information may include generating an artificial intelligence-based learning model capable of determining the fingerprint type.

First, the computing device 100 collects fingerprint-related data samples (S300), and then performs a data classification step (S310) of classifying the samples into at least 12 types of fingerprints through image analysis of the data samples.

In the embodiment of the present invention, the data sample is image data including fingerprints of fingers, and may be an image including fingerprints of five fingers for each of the left and right hands.

In the embodiment of the present invention, image analysis of the data sample may mean analyzing the numbers of nuclei, triangular points, and ridges constituting the fingerprint as shown in FIG. 4 to determine the fingerprint type, while using the data sample including the fingerprint.

The step of classifying the samples into 12 types of fingerprints according to an embodiment of the present invention will be described. First, through executing the program in the computing device, it is determined whether the fingerprint shape is Arch, Loop, Whorl, and Composite patterns through comparative analysis with four types of pre-stored reference shape data.

Next, the above four fingerprint patterns may be further classified as follows: in the case of the Arch, it is determined which one of Simple Arch, Tented Arch, and Loop in Arch through comparison between three detailed reference shape data and the fingerprint shape; in the case of the Loop, it is determined which one of Ulnar Loop and Radial Loop through comparison between two detailed reference shape data and the fingerprint shape; in the case of the Whorl, it is determined which one of Spiral and Concentric (Circle) through comparison between two detailed reference shape data and the fingerprint shape; and in the case of the Composite, it is determined which one of Double Loop, Spiral Double Whorl, Concentric Double Loop, Peacocks Eye, and Radial Peacocks Eye through comparison between five detailed reference shape data and the fingerprint shape. Due to this determination method, image data corresponding to the fingerprint may be classified into 12 types of fingerprints.

Next, the data samples are propagated through the transformation of the data classified into each of the 12 types of fingerprints to generate a training dataset for each fingerprint type (S320). Herein, the propagation may mean rotation of the data sample at various angles, various blur processing and the like.

Thereafter, convolutional neural networks (CNN) training is performed on the training dataset to extract feature points for each type, a pattern for each type is generated through statistical analysis of the extracted feature points, the fingerprint type is determined based on the generated pattern, and then a learning model for determining a fingerprint type, which can determine the fingerprint type, is generated (S330). Herein, the statistical analysis may mean generating a pattern of each fingerprint type by calculating an average change degree, standard deviation, and variance of the feature points.

In particular, in the embodiment of the present invention, the learning on the training dataset may be performed in such a way to calculate fingerprint type discrimination factors such as the pattern of each finger, the number of left and right ridges between the nucleus and the triangular point, a distance between the nucleus and the triangular point, that is, a density, and generate a weight for each element for the determination of each fingerprint type. Accordingly, the learning model for determining the fingerprint type may be generated in a form in which the weights are assigned to each fingerprint type determining element.

Next, data for generating information such as propensity and temperament according to the fingerprint type, for example, academic literature, data, etc. are collected, and then determination data for each fingerprint type is generated based on the collected data (S340). In this case, data collection may be performed through crawling of various information distributed on a plurality of academic literature providing sites and the Internet.

In addition, the generation of determination data is performed through analysis of the data collected through crawling, and a database 230 for determination may be established using the generated determination data.

Next, when a user fingerprint is input from various user input devices 150 (S350), it is determined what the type of user fingerprint is based on the learning model for determining the fingerprint type (S360), and as shown in FIG. 5, a report including the user propensity information is generated using the determination data corresponding to the determined type, then the generated report is provided to the user (S370).

In particular, the report provided in the embodiment of the present invention may further include additional information such as a recommended job according to the user propensity, job suitability, advice for forming a relationship with other propensities, learning direction, and the like, as well as the user propensity information.

To this end, in the embodiment of the present invention, data studied for characteristics according to propensities are collected from online and offline sources, then the first relational database 210 between various propensities is established through analysis of the collected data, and the second relational database 220 in which the propensities are compared and analyzed with the characteristics of jobs and occupational groups may be established. Through this, when the user propensity information is generated based on the fingerprint, the user propensity information and the data in the first and second relational databases 210 and 220 are subjected to linkage analysis to generate additional information, and then a report including the additional information may be generated.

In the embodiment of the present invention as described above, although the inventive method has been described as an example in which the data samples are analyzed by dividing them into 12 types of fingerprints, the data samples may be analyzed by further including sentences that require thinking.

That is, the data samples may be classified into at least 15 types of fingerprints in a manner of: calculating a degree of deformation through comparison between reference shape data for the Radial Loop and the fingerprint images in the data samples, and then if the degree of deformation is out of a preset threshold range, determining the fingerprint as a deformed Radial Loop; calculating a degree of deformation through comparison between reference shape data for the Whorl and the fingerprint images in the data samples, and then if the degree of deformation is out of the preset threshold range, determining the fingerprint as a deformed Whorl; and calculating a degree of deformation through comparison between reference shape data for the Double Loop and the fingerprint images in the data samples, and then if the degree of deformation is out of the preset threshold range, determining the fingerprint as a deformed Double Loop. Accordingly, it is possible to generate a learning model for determining the fingerprint type using a training dataset generated by receiving the data samples, then classifying them into 15 types, followed by transforming and propagating the classified data samples.

Meanwhile, in the embodiment of the present invention, when new fingerprint data is input, the input fingerprint data is determined through the learning model for determining the fingerprint type, and as a result of the determination, if the fingerprint data does not belong to the 12 types of fingerprints, that is, as a result of comparison the shape of the fingerprint data with the 12 types of fingerprints, if the fingerprint data is out of the preset threshold range, processes of determining the fingerprint as a deformed type of fingerprint, and establishing a dataset for the deformed type of fingerprint are performed. Next, when the datasets for the deformed type of fingerprint are accumulated more than a preset number, the learning model for determining the fingerprint type may be updated so as to determine a new fingerprint type based on the accumulated datasets. In this case, the learning model for determining the fingerprint type determines the fingerprint type based on the closest type among the 12 types of fingerprints. Then, after generating user propensity information based on the determined fingerprint type, a report is provided. In this case, a report including information on the degree of matching between the determined fingerprint type and the fingerprint data may be provided.

In addition, determination data for the new fingerprint type is collected and analyzed through new information collection, for example, crawling of fingerprint-related data distributed online, and is generated based on Myers-Briggs Type Indicator (MBTI) of a user having the new fingerprint type and personality analysis data according to blood type. The database 230 for determination may be updated through the generated data.

The artificial intelligence program 200 according to an embodiment of the present invention includes a learning model for determining the fingerprint type composed of a combination of at least one or more commands, and may have a function of accessing the first and second relational databases 210 and 220 and the database 230 for determination, thus to generate data, that is, a report including user propensity information and additional information based on the fingerprint type determined by the learning model for determining the fingerprint type.

In addition, when the new fingerprint type is checked, the artificial intelligence program 200 according to an embodiment of the present invention may generate a training dataset for the new fingerprint type through the same step as S320 based on the checked results, and update the learning model for determining the fingerprint type using the generated training dataset.

Further, the determination data for the new fingerprint type is generated through step S340 or based on the Myers-Briggs Type Indicator (MBTI) of the user having the new fingerprint type and personality analysis data according to blood type. The database 230 for determination may be updated through the generated data.

According to an embodiment of the present invention as described above, the inventive method has been described as an example in which data according to the fingerprint type is collected and generated through the same when generating the determination data. However, the determination data may be generated using information on the user of the data samples, for example, information such as MBTI, blood type and the like. Specifically, when generating the determination data according to the fingerprint type, the computing device 200 may generate the determination data by not only using the collected fingerprint type data, but also reflecting the MBTI, the personality analysis data according to the blood type of the user and the like.

Further, in the embodiment of the present invention, providing the report to the user may be performed in the form of an SNS account, e-mail address of the user, or text message.

Meanwhile, combinations of the respective blocks in the accompanying block diagram and the respective steps in the flowchart may be performed by computer program instructions. These computer program instructions may be installed in the processor of a general purpose computer, special purpose computer, or other programmable data processing equipment, such that the instructions, executed by the processor of the computer or other programmable data processing equipment, will create means for performing the functions described in the respective blocks of the block diagram.

These computer program instructions may also be stored in a computer-usable or computer-readable recording medium (or memory) which can direct the computer or other programmable data processing equipment, to implement a function in a particular manner, such that the instructions stored in the computer-usable or computer-readable recording medium (or memory) may produce an article of manufacture including instruction means which perform the functions described in the respective blocks of the block diagram.

Further, the computer program instructions may be installed in a computer or other programmable data processing equipment, such that a series of operating steps are performed on the computer or other programmable data processing equipment to create a process executed by the computer, and thereby the instructions which execute the computer or other programmable data processing equipment may also provide steps for performing the functions described in the respective blocks of the block diagram.

In addition, the respective blocks may represent modules, segments, or parts of codes including at least one or more executable instructions for performing specific logical function(s). Moreover, it should be noted that the functions mentioned in the blocks may be performed in different order in several alternative embodiments. For example, two successive blocks may in fact be performed substantially at the same time, or may sometimes be performed in reverse order according to functions thereof.

### DESCRIPTION OF REFERENCE NUMERALS

100: Computing device
150: Input device
200: Artificial intelligence program
210, 220: First and second relational databases
230: Database for determination

What is claimed is:

1. A method of providing user propensity analysis service using artificial intelligence-based fingerprints, wherein the user propensity is analyzed and provided to a user based on the fingerprints by a program which is executed by at least one or more processors and stored in a computing device, the method comprising:
generating a learning model for determining fingerprint types, which is configured to collect data samples including fingerprints through an execution of the program in the computing device, and determine at least 12 types of fingerprints through image analysis and artificial intelligence learning on the collected data samples;
collecting data adapted to determine propensities in relation to the fingerprints, and then establishing determination data for determining the propensities for each of at least 12 or more types of fingerprints based on the determined propensities;
when a fingerprint image of the user is input, applying the input fingerprint image to the learning model for determining the fingerprint type to determine the fingerprint type of the user; and
generating user propensity information using the determined data for the fingerprint type and providing the user propensity information to the user.

2. The method according to claim 1, wherein the step of generating a learning model for determining the fingerprint type comprises:
classifying the data samples into at least 12 or more types of fingerprints;
generating a training dataset for each fingerprint type by propagating the data samples through various changes to the classified data samples of each fingerprint type;
performing CNN training on the training dataset of each fingerprint type to extract feature points for each fingerprint type, and generating a pattern of each fingerprint type through statistical analysis of the extracted feature points; and
when the fingerprint image of the user is input, generating a model for determining the fingerprint type, which is adapted to determine the fingerprint type of the user as any one of the 12 or more types of fingerprints using the pattern.

3. The method according to claim 1, wherein the computing device includes a first relational database adapted to compare relationships between various propensities and a second relational database adapted to compare propensities with characteristics of job and occupational groups, and
the step of providing the user propensity information to the user comprises generating a report further including information on jobs and occupational groups suitable for the propensity of the user through linkage analysis between the user propensity information and data stored in the first and second relational databases, by using a relationship between the user and a user having a different propensity from the user.

4. The method according to claim 1, wherein the 12 types of fingerprints comprises at least one of a Radial Loop, Whorl, and a Double Loop patterns, and
the step of generating a model for determining the fingerprint type comprises:
classifying the data samples into at least 15 types of fingerprints in a manner of: calculating a degree of deformation through comparison between reference shape data for the Radial Loop and the fingerprint images in the data samples, and then if the degree of deformation is out of a preset threshold range, determining the fingerprint as a deformed Radial Loop; calculating a degree of deformation through comparison between reference shape data for the Whorl and the fingerprint images in the data samples, and then if the degree of deformation is out of the preset threshold range, determining the fingerprint as a deformed Whorl; and calculating a degree of deformation through comparison between reference shape data for the Double Loop and the fingerprint images in the data samples, and then if the degree of deformation is out of the preset threshold range, determining the fingerprint as a deformed Double Loop;
generating a training dataset for each fingerprint type by propagating the data samples through various changes to the classified data samples of each fingerprint type;
performing CNN training on the training dataset of each fingerprint type to extract feature points for each fingerprint type, and generating a pattern of each fingerprint type through statistical analysis of the extracted feature points; and
when the fingerprint image of the user is input, generating a model for determining the fingerprint type, which is adapted to determine the fingerprint type of the user as any one of the 15 or more types of fingerprints using the pattern.
